SEQUENCE LISTING (12 sequences)

SEQ ID NO: 1 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 2 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 3 - protein, 648 aa, Homo sapiens [sequence omitted]

SEQ ID NO: 4 - protein, 565 aa, Homo sapiens
MRPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPPGLQPGAGGTPGGPGGGGAPPRYATLEHPFHCPRVLKVPSYLSYKFLGERDCAAPCEPARPDGSMFFSQEETRFARLWILTWSVLCCASTFFTVTTYLVDMQRFRYPERPIIFLSGCYTMVSVAYIAGFVLQERVVCNERFSEDGYRTVVQGTKKEGCTILFMMLYFFSMASSIWWVILSLTWFLAAGMKWGHEAIEANSQYFHLAAWAVPAVKTITILAMGQIDGDLLSGVCFVGLNSLDPLRGFVLAPLFVYLFIGTSFLLAGFVSLFRIRTIMKHDGTKTEKLERLMVRIGVFSVLYTVPATIVIACYFYEQAFREHWERSWVSQHCKSLAIPCPAHYTPRMSPDFTVYMIKYLMTLIVGITSGFWIWSGKTLHSWRKFYTRLTNSRHGETTV

SEQ ID NO: 5 - protein, 666 aa, Homo sapiens
MAMTWIVFSLWPLTVFMGHIGGHSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDLNLAGEPTEGAPVAVQRDYGFWCPRELKIDPDLGYSFLHVRDCSPPCPNMYFRREELSFARYFIGLISIICLSATLFTFLTFLIDVTRFRYPERPIIFYAVCYMMVSLIFFIGFLLEDRVACNASIPAQYKASTVTQGSHNKACTMLFMILYFFTMAGSVWWVILTITWFLAAVPKWGSEAIEKKALLFHASAWGIPGTLTIILLAMNKIEGDNISGVCFVGLYDVDALRYFVLAPLCLYVVVGVSLLLAGIISLNRVRIEIPLEKENQDKLVKFMIRIGVFSILYLVPLLVVIGCYFYEQAYRGIWETTWIQERCREYHIPCPYQVTQMSRPDLILFLMKYLMALIVGIPSVFWVGSKKTCFEWASFFHGRRKKEIVNESRQVLQEPDFAQSLLRDPNTPIIRKSRGTSTQGTSTHASSTQLAMVDDQRSKAGSIHSKVSSYHGSLHRSRDGRYTPCSYRGMEERLPHGSMSRLTDHSRHSSSHRLNEQSRHSSIRDLSNNPMTHITHGTSMNRVIEEDGTSA

SEQ ID NO: 6 - protein, 537 aa, Homo sapiens [sequence omitted]

SEQ ID NO: 7 - protein, 585 aa, Homo sapiens
MARPDPSAPPSLLLLLLAQLVGRAAAASKAPVCQEITVPMCRGIGYNLTHMPNQFNHDTQDEAGLEVHQFWPLVEIQCSPDLRFFLCTMYTPICLPDYHKPLPPCRSVCERAKAGCSPLMRQYGFAWPERMSCDRLPVLGRDAEVLCMDYNRSEATTAPPRPFPAKPTLPGPPGAPASGGECPAGGPFVCKCREPFVPILKESHPLYNKVRTGQVPNCAVPCYQPSFSADERTFATFWIGLWSVLCFISTSTTVATFLIDMDTFRYPERPIIFLSACYLCVSLGFLVRLVVGHASVACSREHNHIHYETTGPALCTIVFLLVYFFGMASSIWWVILSLTWFLAAAMKWGNEAIAGYGQYFHLAAWLIPSVKSITALALSSVDGDPVAGICYVGNQNLNSLRRFVLGPLVLYLLVGTLFLLAGFVSLFRIRSVIKQGGTKTDKLEKLMIRIGIFTLLYTVPASIVVACYLYEQHYRESWEAALTCACPGHDTGQPRAKPEYWVLMLKYFMCLVVGITSGVWIWSGKTVESWRRFTSRCCCRPRRGHKSGGAMAAGDYPEASAALTGRTGPPGPAATYHKQVSLSHV

SEQ ID NO: 8 - protein, 706 aa, Homo sapiens
MEMFTFLLTCIFLPLLRGHSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLGPQKKTEQVQRDIGFWCPRHLKTSGGQGYKFLGIDQCAPPCPNMYFKSDELEFAKSFIGTVSIFCLCATLFTFLTFLIDVRRFRYPERPIIYYSVCYSIVSLMYFIGFLLGDSTACNKADEKLELGDTVVLGSQNKACTVLFMLLYFFTMAGTVWWVILTITWFLAAGRKWSCEAIEQKAVWFHAVAWGTPGFLTVMLLALNKVEGDNISGVCFVGLYDLDASRYFVLLPLCLCVFVGLSLLLAGIISLNHVRQVIQHDGRNQEKLKKFMIRIGVFSGLYLVPLVTLLGCYVYEQVNRITWEITWVSDHCRQYHIPCPYQAKAKARPELALFMIKYLMTLIVGISAVFWVGSKKTCTEWAGFFKRNRKRDPISESRRVLQESCEFFLKHNSKVKHKKKHYKPSSHKLKVISKSMGTSTGATANHGTSAVAITSHDYLGQETLTEIQTSPETSMREVKADGASTPRLREQDCGEPASPAASISRLSGEQVDGKGQAGSVSESARSEGRISPKSDITDTGLAQSNNLQVPSSSEPSSLKGSTSLLVHPVSGVRKEQGGGCHSDT

SEQ ID NO: 9 - protein, 574 aa, Homo sapiens
MRDPGAAAPLSSLGLCALVLALLGALSAGAGAQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGPGGGPTAYPTAPYLPDLPFTALPPGASDGRGRPAFPFSCPRQLKVPPYLGYRFLGERDCGAPCEPGRANGLMYFKEEERRFARLWVGVWSVLCCASTLFTVLTYLVDMRRFSYPERPIIFLSGCYFMVAVAHVAGFLLEDRAVCVERFSDDGYRTVAQGTKKEGCTILFMVLYFFGMASSIWWVILSLTWFLAAGMKWGHEAIEANSQYFHLAAWAVPAVKTITILAMGQVDGDLLSGVCYVGLSSVDALRGFVLAPLFVYLFIGTSFLLAGFVSLFRIRTIMKHDGTKTEKLEKLMVRIGVFSVLYTVPATIVLACYFYEQAFREHWERTWLLQTCKSYAVPCPPGHFPPMSPDFTVFMIKYLMTMIVGITTGFWIWSGKTLQSWRRFYHRLSHSSKGETAV

SEQ ID NO: 10 - protein, 694 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGAGGAGGAAAGAGAAGAGAGGPGGRGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQDGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRSLCTRCCWASKGAAVGGGAGATAAGGGGGPGGGGGGGPGGGGGPGGGGGSLYSDVSTGLTWRSGTASSVSYPKQMPLSQV

SEQ ID NO: 11 - protein, 591 aa, Homo sapiens
MAVAPLRGALLLWQLLAAGGAALEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENATAGPAEPHKGLGMLPVAPRPARPPGDLGPGAGGSGTCENPEKFQYVEKSRSCAPRCGPGVEVFWSRRDKDFALVWMAVWSALCFFSTAFTVLTFLLEPHRFQYPERPIIFLSMCYNVYSLAFLIRAVAGAQSVACDQEAGALYVIQEGLENTGCTLVFLLLYYFGMASSLWWVVLTLTWFLAAGKKWGHEAIEAHGSYFHMAAWGLPALKTIVILTLRKVAGDELTGLCYVASTDAAALTGFVLVPLSGYLVLGSSFLLTGFVALFHIRKIMKTGGTNTEKLEKLMVKIGVFSILYTVPATCVIVCYVYERLNMDFWRLRATEQPCAAAAGPGGRRDCSLPGGSVPTVAVFMLKIFMSLVVGITSGVWVWSSKTFQTWQSLCYRKIAAGRARAKACRAPGSYGRGTHCHYKAPTVVLHMTKTDPSLENPTHL

SEQ ID NO: 12 - protein, 581 aa, Homo sapiens
MQRPGPRLWLVLQVMGSCAAISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNGSDEPTRGSGLFPPLFRPQRPHSAQEHPLKDGGPGRGGCDNPGKFHHVEKSASCAPLCTPGVDVYWSREDKRFAVVWLAIWAVLCFFSSAFTVLTFLIDPARFRYPERPIIFLSMCYCVYSVGYLIRLFAGAESIACDRDSGQLYVIQEGLESTGCTLVFLVLYYFGMASSLWWVVLTLTWFLAAGKKWGHEAIEANSSYFHLAAWAIPAVKTILILVMRRVAGDELTGVCYVGSMDVNALTGFVLIPLACYLVIGTSFILSGFVALFHIRRVMKTGGENTDKLEKLMVRIGLFSVLYTVPATCVIACYFYERLNMDYWKILAAQHKCKMNNQTKTLDCLMAASIPAVEIFMVKIFMLLVVGITSGMWIWTSKTLQSWQQVCSRRLKKKSRRKPASVITSGGIYKKAQHPQKTHHGKYEIPAQSPTCV

(19) United States
(12) Patent Application Publication
(10) Pub. No.: US 2009/0081189 A1
(43) Pub. Date: Mar. 26, 2009

(54) Process for in vitro differentiation of neuronal stem cells or of cells derived from neuronal stem cells

(75) Inventors: Armin Schneider, Heidelberg (DE); Martin H. Maurer, Heildelberg (DE); Robert E. Feldmann, Wiesbaden (DE); Wolfgang Kuschinsky, Heidelberg (DE)

Correspondence Address: FOLEY AND LARDNER LLP;SUITE 500, 3000 K STREET NW, WASHINGTON, DC, 20007, US

(73) Assignee: Axaron Bioscfience Ag

(21) Appl. No.: 10/584,341

(22) PCT Filed: Dec. 23, 2004
(86) PCT No.: PCT/EP2004/014673

§ 371 Date: Apr. 26, 2007

(30) Foreign Application Priority Data
Dec. 23, 2003 (DE) 103 61 444.3

(51) Int. Cl. A61K 39/395; C12N 5/06; G01N 33/53; C12N 9/99; C12N 15/87; A61K 31/35; C12Q 1/02; A61K 35/12

(52) U.S. Cl. 424/130.1; 435/377; 435/7.21; 435/184; 435/29; 424/93.7; 514/456; 435/455

(57) ABSTRACT

The process for in vitro differentiation of neuronal stem cells comprises contacting the cells with a substance which inhibits a reaction of the Wnt signal transduction pathway, and culturing of said cells under conditions which enable said cells to propagate and/or differentiate.

In a preferred embodiment of the process, the neuronal stem cells differentiate into brain cell-like cells.

Adult neuronal stem cells have previously been isolated from various regions of the brain (for review see (Gage F H, 2000, Science, 287, 1433-1438; Ostenfeld T and Svendsen C N, 2003, Adv Tech Stand Neurosurg, 28, 3-89)), inter alia also the hippocampus of the mammalian brain (Eriksson P S et al., 1998, Nat Med, 4, 1313-1317; Gage F H et al., 1995, Proc Natl Acad Sci USA, 92, 11879-11883; Johansson C B et al., 1999, Exp Cell Res, 253, 733-736). These cells, in contrast to embryonic stem cells, no longer have the potential of differentiating into any type of somatic cells (totipotency) but they may differentiate into the various types of cells occurring in the brain (pluripotency). In the process, they are subject to substantial morphological and functional changes (van Praag H et al., 2002, Nature, 415, 1030-1034).

The use of neuronal stem cells enables ethical problems, as they appear with the use of embryonic stem cells in medicine and biotechnology, to be avoided (Heinemann T and Honnefelder L, 2002, Bioethics, 16, 530-543).

Other methods of differentiation and selective concentration of neuronal cells comprise more complicated differentiation protocols (Björklund A and Lindvall O, 2000, Nat Neurosci, 3, 537-544; Björklund A and Lindvall O, 2000, Nature, 405, 892-893, 895; Cameron H A et al., 1998, J Neurobiol, 36, 287-306; McKay R, 2000, Nature, 406, 361-364). Thus, for example, cells in fluorescence-aided cell sorting (FACS) must express specific markers in order to be able to be labeled with a fluorescent antibody and subsequently to be separated from the unlabeled cells while passing through a glass capillary. This type of flow cytometry may also damage the cells.

Other selective cell culture media also result in a low yield of differentiated neurons (Wachs F P, Couillard-Despres S, Engelhardt M, Wilhelm D, Ploetz S, Vroemen M, Kaesbauer J, Uyanik G, Klucken J, Karl C, Tebbing J, Svendsen C, Weidner N, Kuhn H G, Winkler J, Aigner L, High efficacy of clonal growth and expansion of adult neural stem cells. Lab Invest. 2003, 83:949-62. Likewise, differentiation monitoring is often difficult.

The previously described methods of stem cell differentiation or of in vitro differentiation of neuronal stem cells or of cells derived therefrom thus have at least one or more of the following disadvantages:
the processes are not suitable for high throughput
the use of embryonic stem cells causes big ethical problems
the differentiation protocols are complicated
the yield of differentiated cells is low
differentiation monitoring is difficult

It is an object of the invention to eliminate or at least minimize the substantial disadvantages of the known processes.

One solution to the stated object is the process for the in vitro differentiation of neuronal stem cells and of cells derived from neuronal stem cells, comprising (a) contacting the cells with a substance which inhibits a reaction of the Wnt signal transduction pathway, and (b) culturing said cells under conditions which enable said cells to propagate and/or differentiate.

In a preferred embodiment of the process of the invention the neuronal stem cells or the cells derived from neuronal stem cells differentiate into brain cell-like cells.

An important signal pathway for the development and differentiation of cells is the Wnt signal pathway (Gerhart J, 1999, Teratology, 60, 226-239; Peifer M and Polakis P, 2000, Science, 287, 1606-1609, see also FIG. 6). It is responsible in ontogenesis and embryogenesis inter alia for the posterior shift of the neural plate and for the development of the mesencephalon and cerebellum (Sokol S Y, 1999, Curr Opin Genet Dev, 9, 405-410). Moreover, Wnt plays an important part in the specification of neuronal cell types (interneurons) (Muroyama Y et al., 2002, Genes Dev, 16, 548-553) and acts as a factor for the self regeneration of stem cells (Katoh M, 2002, Int J Mol Med, 10, 683-687; Song X and Xie T, 2002, Proc Natl Acad Sci USA, 99, 14813-14818). In embryonic stem cells, inhibition of the Wnt signal pathway results in neuronal differentiation of said cells (Aubert J et al., 2002, Nat Biotechnol, 20, 1240-1245). The Wnt signal pathway has been described to maintain the self regeneration and proliferation of hematopoietic stem cells (Reya T et al., 2003, Nature, 423, 409-414; Lako M et al., 2001, Mech Dev, 103, 49-59; Willert K et al., 2003, Nature, 423, 448-452). However, there is to date no knowledge of any effects of Wnt action in stem cells isolated from the adult brain.

The Wnt signal pathway comprises signal chains regulated in a complex manner (Gerhart J, 1999, Teratology, 60, 226-239). Binding of a Wnt signal molecule to the specific receptor results in an inhibition of the signal mediator Dsh (Dishevelled) which in turn inhibits glycogen synthase kinase 3 (GSK-3) (Woodgett J R, 2001, Sci STKE, 2001, RE12). The latter, interacting with axin and APC (adenomatous polyposis coli protein) (Kielman M F et al., 2002, Nat Genet, 32, 594-605), phosphorylates the transcription cofactor beta-catenin which, in its unphosphorylated state, can influence nuclear transcription via the transcription factor Tcf/Lef1. In contrast, phosphorylated beta-catenin is ubiquitinated and degraded in the proteasome.

In another preferred embodiment of the process of the invention, a reaction of the Wnt signal transduction pathway is inhibited by way of inhibition of glycogen synthase kinase 3. This may be caused by the inhibitor genistein.

As an option, it is possible to determine the concentration of β-catenin, a protein of the Wnt signal transduction pathway, and (in the phosphorylated state) product of glycogen synthase kinase 3. The concentration may then be compared to the corresponding concentration of the protein in an untreated comparative cell.

Further embodiments of the invention relate to cells obtainable by a process of the invention, to a neurological tissue replacement having said cells and to pharmaceutical agents (medicaments) containing said cells.

Moreover, the present invention relates to screening processes for identifying substances which inhibit the Wnt signal transduction pathway and which are thus suitable for differentiation of neuronal stem cells and of cells derived from neuronal stem cells, and to medicaments containing said substances.

All of the medicaments of the invention may be used for the treatment of a multiplicity of disorders on which the modulation of the activity or amount of a protein of the Wnt signal transduction pathway may have a beneficial influence. Said diseases include especially disorders which, either directly or indirectly, cause brain cells to die.

The invention further relates to the use of neuronal stem cells which either express a protein capable of inhibiting a reaction of the Wnt signal transduction pathway or do not express a protein of this metabolic pathway or express said protein in an inactive form or at a reduced level, for in vitro differentiation of neuronal stem cells and of cells derived from neuronal stem cells.

The invention furthermore relates to kits for in vitro differentiation of neuronal stem cells and of cells derived from neuronal stem cells.

The term “differentiation” refers in accordance with the present invention to the, in comparison with the starting cell, increasing acquisition or possession of one or more characteristics or functions.

The term “stem cell” characterizes a cell which proliferates, regenerates itself and maintains the ability to differentiate. This also includes progenitor cells. The term “neuronal stem cell” is used for a cell isolated from the central nervous system, which is capable of proliferating, self generating and differentiating with generation of brain cell phenotypes. In this case, a “cell derived from neuronal stem cells” is a brain cell-like cell which nevertheless still has the potential of differentiation and which has been produced from a (hypothetical) neuronal stem cell.

The neuronal stem cells and cells derived from neuronal stem cells here are preferably mammalian cells, said term also including monkeys, pigs, sheep, rats, mice, cattle, dogs etc. Preference is given to the mammal being a human being. The cells used may be fresh or may have been frozen previously or may originate from an earlier culture.

The cells are cultured in a suitable medium. Various media are commercially available, including neurobasal medium, DMEM (Dulbecco's Modified Eagle's Medium), ex vivo serum-free medium, Iscove's medium, etc. Suitable antibiotics (e.g. penicillin and streptomycin) may be added to prevent bacterial growth and other supplements such as heparin, glutamine, B27, EGF, FGF2 or fetal calf serum may be added.

After inoculating the medium, the cultures are cultured under standard conditions, usually at 37° C. in a 5% CO₂ atmosphere. Fresh medium may be supplied in a suitable manner, partly by removing part of the medium and replacing it by fresh medium. A large variety of commercial systems have been developed in order to remove disadvantageous metabolic products during culturing of mammalian cells. The use of these systems enables the medium to be maintained as continuous medium so that the concentration of various ingredients remains relatively constant or within a predefined range.

The Wnt signal transduction pathway is known to the skilled worker (Gerhart J, 1999, Teratology, 60, 226-239; Peifer M and Polakis P, 2000, Science, 287, 1606-1609, see also FIG. 6). Further reaction steps of the Wnt signal transduction pathway, further receptors influencing said signal transduction pathway or new proteins involved in the already known reaction steps are likewise to be regarded as part of the Wnt signal transduction pathway for the purposes of the present invention.

“To inhibit” or “inhibition” is to be interpreted broadly in context with the modulation of a reaction of the Wnt signal transduction pathway and comprises partial, essentially complete or complete stopping or blocking, based on very different cell-biological mechanisms, of a reaction of said signal transduction pathway. In this context, it is statistically probable to be able to recognize a significant difference to the corresponding reaction of an untreated comparative cell.

The skilled worker is familiar with a large variety of strategies in order to influence said reactions in the desired way. Preference is given according to the invention to a strategy consisting of the use of a substance which inhibits a protein itself of the Wnt signal transduction pathway or which specifically reduces a substantial property of said protein. Corresponding substances are known to the skilled worker, for example substrate analogs which compete with the original substrate but are converted only to a small extent or not at all, thus blocking the particular enzyme. Furthermore, such a substance could also be an antibody. Another procedure according to the invention comprises the use of an antisense nucleic acid which is fully or partially complementary to at least part of a sense strand of a nucleic acid coding for a protein of the Wnt signal transduction pathway. The preparation of antisense nucleic acids of this kind in a biological or enzymic/chemical way is familiar to the skilled worker. In another embodiment, an appropriate inhibition may also take place by way of influencing regulatory elements, for example by specific DNA-binding factors which modulate expression of the target gene. Examples of regulatory elements are promoters, enhancers, locus control regions, silencers or in each case parts thereof. Preferably, regulation may also be produced by RNA interference (RNAi) by means of double-stranded RNA.

In a preferred embodiment of the process of the invention, the neuronal stem cells differentiate into brain cell-like cells. “Brain cell-like cells” here are characterized in that they have essential morphological or functional features of brain cells. Such a cell expresses particular marker proteins, for example a neuron-like cell expresses at least one of the marker proteins β₃-tubulin, MAP2a or MAP2b. An astrocyte-like cell expresses GFAP, while an oligodendrocytic cell expresses OCT and/or O4. A brain cell-like cell furthermore has a typical form and its morphology is similar to that of a brain cell, for example due to the typical processes. Neuron-like cells may moreover produce action potentials and have a membrane potential.

The invention moreover relates to another embodiment of the process of the invention, which comprises, as an optional further step, determining the concentration of a protein of the Wnt signal transduction pathway. For this purpose, the amount of protein is quantified and compared with the amount of the same protein in an untreated comparative cell in which no reaction of the Wnt signal transduction pathway has been inhibited.

In a preferred embodiment of the process of the invention, the protein whose concentration is determined is β-catenin. β-catenin is phosphorylated in the course of the Wnt signal transduction pathway, phosphorylated β-catenin is ubiquitinated and degraded in the proteasome. The presence of a relatively large amount of said protein (compared to an untreated comparative sample) thus indicates inhibition of the Wnt signal transduction pathway.

In a further preferred embodiment, the concentration of the protein, in particular of β-catenin, is determined by means of an antibody. A β-catenin-specific antibody is commercially available (Chemicon International, Temecula, USA).

The term “antibody” has, with respect to the present invention, a very broad meaning and includes monoclonal antibodies, polyclonal antibodies, human or humanized antibodies, recombinant antibodies, single chain antibodies, synthetic antibodies and antibody fragments (e.g. Fab, F(ab)₂ and Fᵥ), as long as they have the desired biological activity. The antibodies or fragments may be used alone or in mixtures. The production of said antibodies is familiar to the skilled worker. For the purposes of detection, such an antibody will preferably be labeled with a detectable compound.

Preference is given to the reaction of the Wnt signal transduction pathway being inhibited by way of inhibition of glycogen synthase kinase 3. Particular preference is given here to inhibition of glycogen synthase kinase 3 beta.

Inhibition means also in this context partial, essentially complete or complete stopping or blocking, based on a wide variety of cell biological mechanisms, of a reaction of said signal transduction pathway and is to be interpreted broadly.

One or more inhibitor(s) for inhibition of glycogen synthase kinase 3 may preferably be selected from the group consisting of kinase inhibitors, estrogen analogs, phytoestrogens, corticoids or salts, in particular 4-benzyl-2-methyl-1,2,4-thiazolidine-3,5-dione, 2-thio(3-iodobenzyl)-5-(1-pyridyl)-[1,3,4]-oxadiazole, 3-(2,4-dichlorophenyl)-4-(1-methyl-1H-indol-3-yl)-1H-pyrrole-2,5-dione, 3-[(3-chloro-4-hydroxyphenyl)amino]-4-(2-nitrophenyl)-1H-pyrrole-2,5-dione, lithium salts and beryllium salts. Moreover, it is also possible for alkali metals or alkaline earth metals to act as inhibitors. It is further possible to use modified forms of the abovementioned inhibitors.

In another preferred embodiment of the process of the invention, genistein (4′,5,7-trihydroxyisoflavone) is used as appropriate inhibitor of glycogen synthase kinase 3.

In this case, genistein is used in a concentration suitable for inhibition, preferably in a concentration of 10-250 μmol/l, particularly preferably in a concentration of 40-60 μmoll. Less preference is given to a concentration of from 250 μmol to 1 mmol.

Preferably, the reaction of the Wnt signal transduction pathway may also be inhibited by at least one antagonist of the “Frizzled” receptor.

According to the present invention, “antagonist” refers to a substance which may displace active physiological transmitters or analogs thereof from a receptor but which is not capable of causing a physiological reaction and signal transduction, thus blocking said receptor.

An alternative way of developing the antagonists of the invention is rational drug design (Böhm, Klebe, Kubinyi, 1996, Wirkstoffdesign [Drug design], Spektrum-Verlag, Heidelberg, Germany). Here, the structure or partial structure of the receptor is utilized in order to find, by means of molecular modeling programs, structures for which high affinity to the receptor can be predicted. These substances are synthesized and then assayed for their action.

Preferably, the at least one antagonist of the Frizzled receptor may be selected from the group consisting of secreted Frizzle-related proteins (sFRP), Dickkopf (Dkk), Wnt, Fzd, Frat, Nkd, VANG1/STB2, ARHU/WRCH₁, ARHV/WRCH₂, GIPC2, GIPC3, betaTRCP2/FBXW1B, SOX17, TCF-3, WIF-1, Cerberus, Sizzled, Crescent, Coco, Soggy, Kremen and low-density-lipoprotein-receptor-related proteins (LRP).

In another preferred embodiment of the invention, the cells derived from neuronal stem cells, which are used as “starting point” of the process, are cells selected from the group consisting of neuroblastoma cells, PC12 cells, cells of neuronal primary cultures and 293 cells.

The invention further relates to cells which have been treated (are obtainable) by any of the processes of the invention and to a neurological tissue replacement comprising such cells. For this purpose, cells isolated from a patient by biopsy are grown by any of the processes of the invention and then reimplanted into this or another patient. It is also possible to use cells of mammals other than humans for this purpose, for example cells of monkeys, pigs, sheep, rats, mice, cattle, dogs, etc. The transplantation of in vitro differentiated embryonic cells is an established process. Undifferentiated neuronal progenitors have also been transplanted previously.

In addition it is possible to influence the growth behavior of adult neuronal stem cells in vivo by the described embodiments of the medicaments of the invention.

The invention further relates to (screening) processes for finding and identifying substances which inhibit the Wnt signal pathway and are suitable for differentiation of neuronal stem cells or of cells derived from neuronal stem cells. A process of this kind may comprise the following steps:
(c) contacting said cells with said substance,
(d) determining the β-catenin concentration in said cells,
(e) comparison with a suitable comparative cell, and
(f) detecting differentiation of said cells.

It is also possible for the purpose of finding these substances to use direct or indirect detection processes familiar to the skilled worker for finding interaction partners. Examples of said processes include:
antibody selection techniques
a number of processes under the generic term “yeast-N-hybrid” systems, for example the yeast-2-hybrid system
phage display systems
immunoprecipitations
immunoassays such as ELISA or Western blot
reporter test systems
screening of libraries of low-molecular weight compounds
molecular modeling using structural information of the Wnt signal transduction proteins
microarray
protein array
antibody array
mass spectrometry or HPLC-based screening systems.

The interaction partners found in these processes are then examined for their ability to inhibit the Wnt signal pathway and to cause differentiation of neuronal stem cells.

The invention further relates to the use of medicaments for the treatment or prophylaxis of diseases on which modulation of the activity or amount of a protein of the Wnt signal transduction pathway can have a beneficial influence. Said diseases include in particular disorders or conditions which lead directly or indirectly to the death of brain cells.

The medicaments of the invention may contain here either cells treated by any of the processes of the invention and/or substances which inhibit a reaction of the Wnt signal transduction pathway, in particular inhibitors of glycogen synthase kinase 3 and/or antagonists of the Frizzled receptor and/or antibodies to proteins of the Wnt signal transduction pathway.

The active compounds are administered in a therapeutically active amount which can be determined routinely by a skilled worker in the relevant field of study, according to techniques for determining the dosage range.

Examples of said diseases may be the group of cerebral malformations and cerebral developmental anomalies such as cerebral palsies in infants, craniocervical junction abnormalities or dysraphic syndromes. Said diseases moreover include the group of degenerative and atrophic processes of the brain and the spinal cord, such as senile and presenile atrophies of the brain, for example Alzheimer's disease, Binswanger's disease or Pick's disease. The disorders which can be treated by means of the medicaments of the invention also include basal ganglia disorders such as Huntington's disease and HDL2, chorea, athetosis and dystonia. Mention should furthermore be made of spongioform encephalopathies and also of degenerations of the corticospinal tract and of the anterior horn of the spinal cord, for example amyotrophic lateral sclerosis, spinal muscular atrophy and progressive bulbar paralysis. They may likewise be degenerative ataxias such as Friedreich's disease, Refsum's disease or spinocerebellar ataxias type 1-25. Metabolic and toxic processes of the brain and of the spinal cord, such as hereditary metabolic disorders of the amino acid, lipid, carbohydrate and metal ion metabolisms, in particular Wilson's disease, may also be treated by the medicaments of the invention. Furthermore, multiple sclerosis and demyelinating diseases of the central and peripheral nerve system, brain and spinal cord tumors and traumatic damage to the nerve system may also be listed. Circulation disorders of the brain and of the spinal cord, in particular cerebral infarctions and other forms of stroke, and muscular disorders based on damage to the nerve system, in particular post-traumatic muscular atrophies may be treated by the medicaments of the invention.

Furthermore, preference is given to modifications or formulations of the medicaments of the invention, which increase the ability to pass the blood brain barrier or which shift the distribution coefficient toward the brain tissue. Examples of such modifications are addition of a protein transduction domain (ptd) or of tat sequences. It is also possible to use nuclear localization sequences (NLS) or nuclear translocation sequences (NTS).

Preference is also given to the addition of any substances to the medicaments of the invention, which support the therapeutic action of said medicaments. This effect may be cumulative or over-additive. Examples suitable for this purpose are substances with neuroprotective properties, such as erythropoietin, BDNF, VEGF, CTNF, GCSF and GMCSF and medicaments influencing inflammations.

The medicaments of the invention can be formulated according to the standard processes available in the art. Thus it is possible, for example, to add a pharmaceutically suitable carrier (or excipient). Suitable carriers or excipients are familiar to the skilled worker. Said carrier or excipient may be a solid, semi-solid or liquid material serving as a vehicle or medium for the active component. The skilled worker of average knowledge in the field of preparing compositions can readily select the suitable form and type of administration, depending on the particular properties of the active compound selected, of the disorder to be treated or of the disease state to be treated, on the stage of the disease and on other relevant circumstances (Remington's Pharmaceutical Sciences, Mack Publishing Co. (1990)). The proportion and nature of the pharmaceutically acceptable carrier or excipient are determined by the solubility and chemical properties of the active compound selected.

Particular preference is given to administering the medicaments by direct intercerebral injection into the brain or as intraventricular injection. They may preferably also be administered intravenously, as a tablet or as a nasal spray. A gene transfer by modified adenoviruses is also a preferred subject matter of the invention.

The invention further also relates to a process for finding and identifying substances (screening process) for detecting brain cell-like cells and brain cells, comprising the steps
(i) determining the concentration of β-catenin, and
(ii) comparing the concentration determined from (i) with the β-catenin concentration of a suitable comparative cell.

Here too, a cell which has not been treated with the corresponding substance may again be used as comparative cell. In a particular embodiment of the process of the invention, the β-catenin concentration is determined by means of an antibody.

The invention further relates to the use of β-catenin as diagnostic marker for identifying brain cell-like cells and brain cells. Said detection may also be carried out inter alia by means of an antibody.

The invention further relates to a recombinant, neuronal stem cell or to a cell derived from a neuronal stem cell. These cells contain a nucleic acid construct coding for a polypeptide which results in inhibition of a reaction of the Wnt signal transduction pathway. The cells are used for in vitro differentiation of the stem cells into brain cell-like cells.

In this connection, the nucleic acid construct includes a nucleic acid coding for a protein with inhibitory action under the control of a promoter. The promoter here may be any known promoter which is active in the host cell into which the nucleic acid construct is to be introduced, i.e. which activates transcription of the downstream protein in said host cell. The promoter here may be a constitutive promoter which expresses the downstream protein continuously or may be a nonconstitutive promoter which expresses only at defined times in the course of development or under certain circumstances.

The nucleic acid construct of the invention may, where appropriate, contain further control sequences. A control sequence means any nucleotide sequence which influences expression of the inhibitory polypeptide, such as, in particular, the promoter, an operator sequence, i.e. the DNA-binding site for a transcription activator or a transcription repressor, a terminator sequence, a polyadenylation sequence or a ribosome binding site.

The nucleic acid construct of the invention may moreover contain a nucleic acid sequence which can be used by the vector to replicate in the host cell in question. Such nucleotide sequences are usually referred to as “origin of replication”. An example of such nucleotide sequences is the SV40 origin of replication which is employed in mammalian host cells.

The nucleic acid construct may furthermore contain one or more selection markers. A selection marker is a gene which is under the control of a promoter and which encodes a protein complementing a physiological defect of the host cell. Particular selection markers are the gene coding for dihydrofolate reductase (DHFR) or else a gene causing resistance to antibiotics such as, in particular, ampicillin, kanamycin, tetracycline, blasticidin, gentamycin, chloramphenicol, neomycin or hygromycin.

A large number of recombinant vectors for expressing a target protein in host cells have been disclosed in the prior art and many of them are also commercially available.

Moreover, the protein with inhibitory action may also be expressed as fusion protein. In this case, a number of amino acids are added N- or C-terminally to the protein to be expressed. Said amino acids may have, for example, the function of increasing expression of the recombinant protein, improving its solubility, facilitating its purification or enabling its detectability.

Furthermore, the cell may have been transfected stably or transiently with the nucleic acid construct.

Transfection or transformation means any kind of process which may be used for introducing a nucleic acid sequence into an organism. A multiplicity of methods is available for this process (see also Sambrook et al., Molecular cloning: A Laboratory Manual., 2nd ed., Cold Spring Harbor Laboratory Press, Cold Spring Harbor, N.Y., 1989).

Transient transformation means introducing a nucleic acid construct into a cell, with said nucleic acid construct not integrating into the genome of the transformed cell. In contrast, in a stable transformation, the nucleic acid construct or parts of said construct is or are integrated into the genome of the transformed cell.

The invention further relates to differentiation of a recombinant, neuronal stem cell in which at least one protein of the Wnt signal transaction pathway is not expressed, is expressed inactively or is expressed at a reduced level in comparison with the corresponding wild type stem cell into brain cell-like cells.

Preference is given here to at least one gene coding for a protein of the Wnt signal transduction pathway or a DNA section involved in expression of said gene being completely or partially deleted or having a mutation.

“Mutations” here comprise substitutions, additions or deletions of one or more nucleotides. “Substitution” means the replacement of one or more nucleotides with one or more nucleotides. “Addition” refers to the addition of one or more nucleotides. “Deletion” is the removal of one or more nucleotides.

The invention further relates to a kit for in vitro differentiation of neuronal stem cells and of cells derived from neuronal stem cells, comprising a recombinant, neuronal stem cell which comprises a nucleic acid construct for expressing a protein capable of inhibiting a reaction of the Wnt signal transduction pathway.

The invention furthermore relates to a kit for in vitro differentiation of neuronal stem cells and of cells derived from neuronal stem cells, comprising a recombinant, neuronal stem cell in which at least one protein of the Wnt signal transduction pathway is not expressed, is expressed inactively or is expressed at a reduced level in comparison with the corresponding wild type stem cell.

For cells contained in both kits have been described in detail previously. Said kits may further comprise other elements and substances, such as experimental instructions, media, media supplements, etc.

The process of the invention is explained in more detail by the drawing:

in which

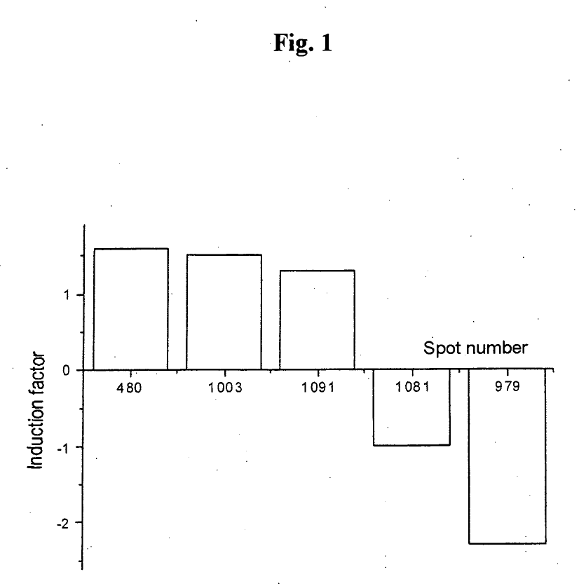

FIG. 1 depicts the semi-quantitative alterations of proteins of the Wnt signal transduction pathway before and after and after the differentiation protocol. For this purpose, a protein extract from adult neuronal stem and progenitor cells was separated by isoelectric point (1st dimension) and molecular weight (2nd dimension). Identified protein spots of the Wnt signal pathway were excised for identification and examined by mass spectrometry,

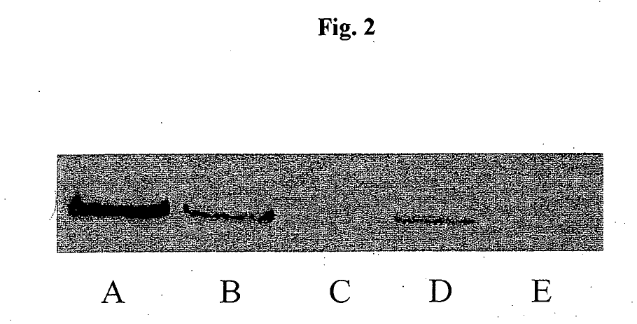

FIG. 2 depicts results of the functional analysis of the Wnt signal pathway in differentiated and undifferentiated adult neuronal stem and progenitor cells with the aid of a Western blot. Beta-catenin was made visible in the protein extracts from adult neuronal stem and progenitor cells by means of specific antibodies. (A) depicts results for undifferentiated cells, without blockage of the Wnt signal pathway, (B) for undifferentiated cells, with blockage of the Wnt signal pathway by genistein, (C) for the negative control, (D) for differentiated cells, without blockage of the Wnt signal pathway, (E) differentiated cells, with blockage of the Wnt signal pathway by genistein,

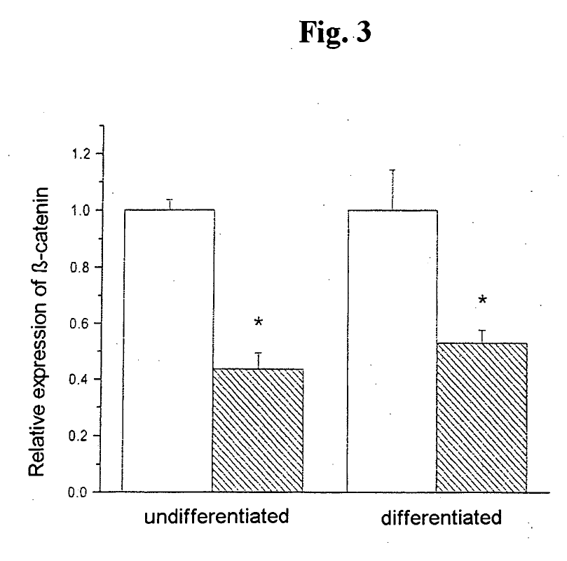

FIG. 3 depicts a semi-quantitative representation of the result of FIG. 3. After addition of genistein, expression of β-catenin can be reduced by approximately a factor of 2,

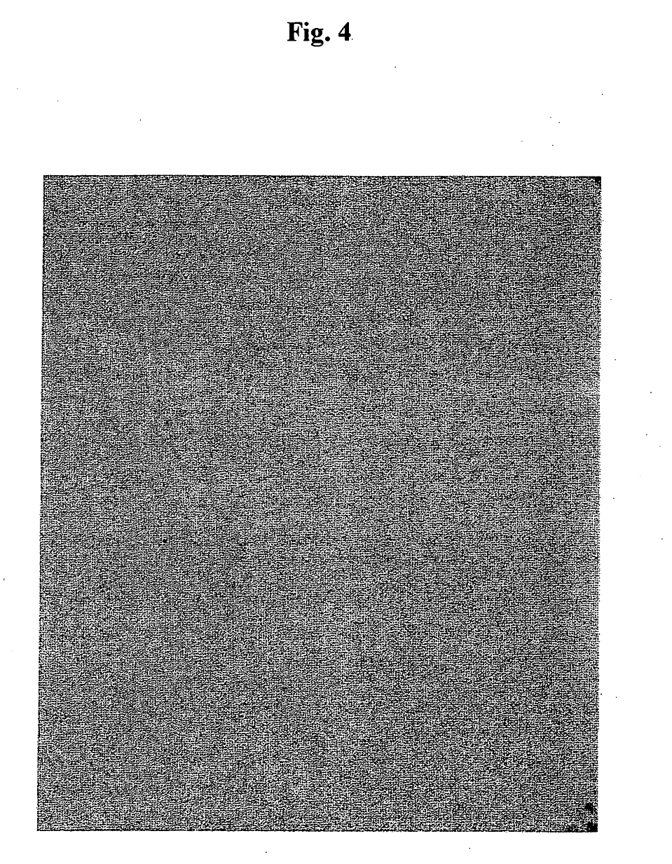

FIG. 4 depicts differentiated neuronal stem and progenitor cells in the cell culture after the differentiation protocol, and

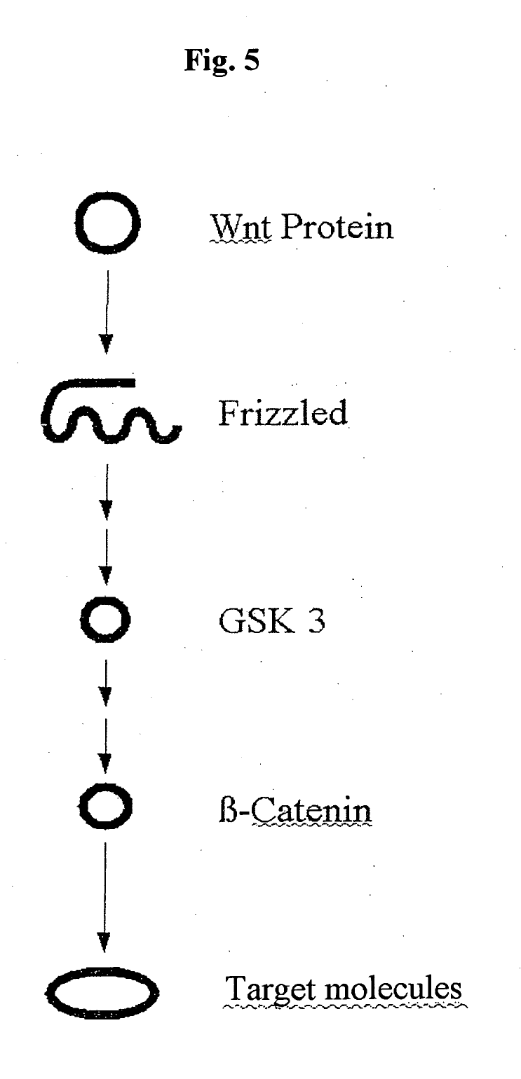

FIG. 5 depicts a diagrammatic representation of the Wnt signal transduction pathway.

### EXAMPLE 1

### Identification of the Wnt Signal Pathway in Neuronal Stem and Progenitor Cells

A protein extract is isolated from cultured neuronal stem and progenitor cells, and proteins of the Wnt signal pathway are identified therein by two-dimensional gel electrophoresis.

Neuronal stem cells are isolated from the hippocampus, olfactory bulb and subventricular zone of the brain of 4-6 week old rats in a process known to the skilled worker (Gage F H et al., 1995, Proc Natl Acad Sci USA, 92, 11879-11883; Gage F H et al., 2000, WO2000047718A1; Ray J et al., 1993, Proc Natl Acad Sci USA, 90, 3602-3606; Reynolds B A and Weiss S, 1992, Science, 255, 1707-1710; Weiss S et al., 1994, WO1994009119A1). For this purpose, the brains were removed and washed in 50 ml of ice cold Dulbecco's and phosphate-buffer saline (DPBS) supplemented with 4.5 g/l glucose (DPBS/Clc). Said brain regions from 6 animals are dissected, washed in 10 ml of DPBS/Glc and centrifuged at 1600 g and 4° C. for 5 min. The supernatant is removed and subsequently the tissue is cut up mechanically. The tissue pieces are washed with DPBS/Glc medium at 800 g for 5 min and the three pellets are resuspended in 0.01% (w/v) papain, 0.1% (w/v) Dispase II (neutral protease), 0.01% (w/v) DNase Iand 12.4 mM MnSO₄ in Hank's balanced salt solution (HBSS). The tissue was triturated using plastic pipette tips and incubated at room temperature for 40 min, mixing the solution every 10 min. The solution is centrifuged at 800 g and 4° C. for 5 min and the pellets are washed three times in 10 ml of DMEM Ham's F-12 medium supplemented with 2 mM L-glutamine, 100 IU/ml penicillin and 100 IU/ml streptomycin. The pellets are resuspended in 1 ml of neurobasal medium supplemented with B27 (Invitrogen, Karlsruhe), 2 mM L-glutamine, 100 IU/ml penicillin and 100 IU/ml streptomycin, 20 ng/ml endothelial growth factor (EGF), 20 ng/ml fibroblast growth factor 2 (FGF-2) and 2 μg/ml heparin. The cells are introduced at a concentration of 25 000-100 000 cells/ml into suitable culture dishes (BD Falcon, Heidelberg, Germany) under sterile conditions. The culture dishes are incubated in a 5% CO₂ atmosphere at 37° C. The culture medium is changed once per week, with about two-thirds being replaced and one third being retained as conditioned medium.

For two dimensional gel electrophoresis, the stem and progenitor cells, after 5 passages of in each case approximately 14 days, are washed 3 times in 300 mosmol/l Tris-HCl sucrose, pH 7.4, and lysed in a sample buffer consisting of 7 M urea, 2 M thiourea, 4% (w/v) CHAPS, 0.5% (v/v) Triton X-100, 0.5% (v/v) IPG buffer pH 3-10 (Amersham Biosciences, Uppsala, Sweden), 100 mM DTT and 1.5 mg/mL complete protease inhibitor (Roche, Mannheim, Germany) in an orbital shaker at room temperature for 1 hour. The lysate is then centrifuged at 21 000×g for 30 min and the protein content of the supernatant is determined by the Bradford method (Bradford M M, 1976, Anal Biochem, 72, 248-254).

Two-dimensional gel electrophoresis is carried out according to standard protocols (Görg A et al., 2000, Electrophoresis, 21, 1037-1053). Samples of 500 μg are applied to nonlinear pH 3-10 gradient IEF gel strips of 18 cm in length for isoelectric focussing (Amersham Bioscience, Freiburg, Germany). After swelling at 30 V for 12 h, 200 V, 500 V, and 1000 V are applied for 1 hour each. The voltage is then increased to 8000 V and kept constant for 12 h. This produces 100 300 Vh on the IPGphor IEF system (Amersham Bioscience, Freiburg, Germany) for isoelectric focussing. Separation in the second dimension is carried out in 12.5% polyacrylamide gels in the presence of 10% SDS. To the gels (180×200×1.5 mm³) 30 mA are applied for 30 min and 100 mA are applied for approximately 4 h in a water-cooled vertical electrophoresis chamber (OWL Scientific, Woburn, Mass., USA). In order to make the proteins visible, the gels are stained with silver nitrate according to a modified protocol (Blum H et al., 1987, Electrophoresis, 8, 93-99). This method is compatible with a subsequent mass spectrometry. The gels are then scanned in and the images are measured densitometrically using the special software Phoretix 2D Professional (Nonlinear Dynamics Ltd., Newcastle-upon-Tyne, UK). After correcting for background, the protein spots of the Wnt signal pathway are measured according to optical density and volume. The proteins are identified by mass spectrometry (Proteosys AG, Mainz, Germany) (FIG. 1).

### EXAMPLE 2

### Detection of Regulation of the Identified Proteins in Neuronal Stem and Progenitor Cells by Differentiation In Vitro

Differentiation of the adult neuronal stem cells is caused by removing the EGF and bFGF growth factors from the medium and adding fetal calf serum (FCS). For this purpose the cells are removed from the culture dishes, centrifuged in culture medium at 800 g and 4° C. for 10 min and are washed three times in 10 ml of DPBS at 800 g and 4° C. The cells are separated enzymatically and resuspended in a new culture dish in 4 ml of neurobasal medium supplemented with B27 (Invitrogen, Karlsruhe, Germany), 2 mM L-glutamine, 100 U/ml penicillin and 100 IU/ml streptomycin and 2 μg/ml heparin. The medium is additionally supplemented with 5% fetal calf serum. The cells were introduced at a concentration of 25 000-100 000 cells/ml into suitable culture dishes (BD Falcon, Heidelberg) under sterile conditions. The culture dishes are incubated at 37° C. in a 5% CO₂ atmosphere for two days.

The in vitro differentiated cells are studied by means of two-dimensional electrophoresis (see above, example 1) and the results for the optical densities of the protein spots are compared to those for undifferentiated cells, using statistical test methods. For this purpose, a Student's t-test is used, with a significance level of p<0.05 being considered statistically significant. As a result, the proteins Pontin 52, proteasome subunit alpha-1 and proteasome subunit alpha-6 (table 1) were identified as being expressed in a regulated manner (FIG. 2).

| | Theoret- | Theoret- | Experi- | | Method of | Regulation | Induc- | Induc- |
|---|---|---|---|---|---|---|---|---|
| | ical | ical | mental | Mascot | identifi- | [%, +supreg, | tion | tion |
| GenBank annotation | pl | MW (Da) | MW (Da) | score | cation | in undiff] | factor | factor |
| RuvB-like protein 1; Pontin 52 | 6.02 | 50524 | 52000 | 176 | MALDI-TOF | 59.5 | 1.6 | 1.6 |
| adenomatosis polyposis coil binding protein Eb1 | 5.02 | 30168 | 31000 | 65 | MALDI-TOF | −56.0 | 0.4 | −2.3 |
| proteasome (prosome, macropain) subunit, alpha | 5.14 | 29784 | 30000 | 77 | MALDI-TOF | 47.8 | 1.5 | 1.5 |
| type 1 | | | | | | | | |
| expressed sequence C67272 | 5.12 | 23450 | 27000 | 129 | MALDI-TOF | −0.3 | 1.0 | −1.0 |
| proteasome (prosome, macropain) subunit, alpha | 6.35 | 27838 | 27000 | 97 | MALDI-TOF | 30.4 | 1.3 | 1.3 |
| type 6 | | | | | | | | |

### EXAMPLE 3

### Detection of β-Catenin Regulation after Differentiation and Inhibition of the Wnt Signal Pathway

To inhibit the Wnt signal pathway, the unspecific kinase inhibitor genistein is added at a concentration of 50 μM in order to inhibit the action of glycogen synthase kinase 3 (GSK 3) (Murase S et al., 2002, Neuron, 35, 91-105).

Subsequently, a protein extract (see above, example 1) is prepared and the beta-catenin protein is identified by one dimensional gel electrophoresis and Western blotting (FIG. 3, FIG. 4).

The protein extracts of the adult neuronal stem cells are first fractionated in a 12% polyacrylamide gel in Lammli buffer consisting of 2% (w/v) sodium dodecylsulfate, 10% (v/v) glycerol, 100 mM dithiothreitol, 60 mM Tris-HCl, pH 6.8, 0.001% bromophenol blue and 5% 2-mercaptoethanol and applied to a nitrocellulose membrane (Optitran BA-S83, 0.2 μm, Schleicher & Schnell, Dassel, Germany) by the semi-dry blotting method (Kyhse-Andersen J, 1984, J Biochem Biophys Methods, 10, 203-209). The membrane is incubated with a suitable reagent in order to suppress unspecific and antibody binding reactions, incubated for 1 h (Seablock, Pierce, Rockford, Ill., USA) and then incubated with the primary antibody (beta-catenin, 1:5000, BD Biosciences, Heidelberg, Germany) in TBST comprising 60 mM NaCl, 100 mM Tris-HCl, pH 7.5 and 0.1% (v/v) Tween 20 at 4° C. overnight. On the following day, the membranes are washed in TBST for 3×5 min and the secondary antibody (ImmunoPure Rabbit Anti-Mouse IgG, (H+L), Peroxidase Conjugated, Pierce, Rockford, Ill., USA) is applied in a 1:20 000 dilution in TBST for 2 h. Antibody binding is detected by way of chemiluminescent signals. Imaging of the chemiluminescent signals on X-ray films is carried out for 30 s using a suitable substrate (SuperSignal West Pico, Pierce, Rockford, Ill., USA). The X-ray films are developed and measured densitometrically. The results for undifferentiated cells without inhibition of the Wnt pathway, undifferentiated cells with inhibition of the Wnt pathway, differentiated cells without inhibition of the Wnt pathway and differentiated cells with inhibition of the Wnt pathway were compared (FIG. 4). Beta-catenin expression in cells with inhibition of the Wnt pathway was found to be reduced by approximately a factor of two.

1-31. (canceled)

32. A process for the in vitro differentiation of neuronal stem cells and of cells derived from neuronal stem cells, comprising
(a) contacting the cells with a substance which inhibits a reaction of the Wnt signal transduction pathway, and
(b) culturing said cells under conditions which enable said cells to propagate and/or differentiate.

33. The process as claimed in claim 32, characterized in that the cells differentiate into brain cell-like cells.

34. The process as claimed in claim 32, characterized in that, where appropriate, a step (c) comprises determining the concentration of a protein of the Wnt signal transduction pathway.

35. The process as claimed in claim 34, characterized in that the protein concentration is determined by means of an antibody.

36. The process as claimed in claim 35, characterized in that the protein is β-catenin.

37. The process as claimed in claim 32 characterized in that the reaction of the Wnt signal transduction pathway is inhibited by way of inhibition of glycogen synthase kinase 3.

38. The process as claimed in claim 37, characterized in that glycogen synthase kinase 3 is inhibited by at least one inhibitor selected from the group consisting of kinase inhibitors, estrogen analogs, phytoestrogens, corticoids and salts, in particular 4-benzyl-2-methyl-1,2,4-thiazolidine-3,5-dione, 2-thio(3-iodobenzyl)-5-(1-pyridyl)-[1,3,4]-oxadiazole, 3-(2,4-dichlorophenyl)-4-(1-methyl-1H-indol-3-yl)-1H-pyrrole-2,5-dione, 3-[(3-chloro-4-hydroxyphenyl)amino]-4-(2-nitrophenyl)-1H-pyrrole-2,5-dione, lithium salts, beryllium salts.

39. The process as claimed in claim 38, characterized in that the inhibitor is genistein.

40. The process as claimed in claim 39, characterized in that genistein is used in a concentration of 10-250 μmol/l.

41. The process as claimed in claim 32, characterized in that the reaction of the Wnt signal transduction pathway is inhibited by at least one antagonist of the Frizzled receptor.

42. The process as claimed in claim 41, characterized in that the at least one antagonist is selected from the group consisting of secreted Frizzle-related proteins (sFRP), Dickkopf (Dkk), Wnt, Fzd, Frat, Nkd, VANG1/STB2, ARHU/WRCH1, ARHV/WRCH₂, GIPC2, GIPC3, betaTRCP2/FBXWIB, SOX17, TCF-3, WIF-1, Cerberus, Sizzled, Crescent, Coco, Soggy, Kremen and low-density-lipoprotein-receptor-related proteins (LRP).

43. The process as claimed in claim 32, characterized in that the cells derived from neuronal stem cells are cells selected from the group consisting of neuroblastoma cells, PC12 cells, cells of neuronal primary cultures and 293 cells.

44. A cell obtainable by the process as claimed in claim 32.

45. A neurological tissue replacement comprising cells as claimed in claim 44.

46. A pharmaceutical agent comprising cells as claimed in claim 44.

47. A screening process for identifying substances which inhibit the Wnt signal transduction pathway and are suitable for the differentiation of neuronal stem cells and of cells derived from neuronal stem cells, comprising
(c) contacting said cells with said substance,
(d) determining the β-catenin concentration in said cells,
(e) comparison with a suitable comparative cell, and
(f) detecting differentiation of said cells.

48. A pharmaceutical agent comprising inhibitors of glycogen synthase kinase 3, antagonists of the Frizzled receptor and/or antibodies to proteins of the Wnt signal transduction pathway.

49. The agent as claimed in claim 48, characterized in that the inhibitor of glycogen synthase kinase 3 is genistein.

50. The method of treating a disease on which modulation of the activity or amount of a protein of the Wnt signal transduction pathway may have a beneficial influence comprising administering a pharmaceutical agent as claimed in claim 46 in an amount sufficient to treat the disease.

51. The method of treating a disease on which modulation of the activity or amount of a protein of the Wnt signal transduction pathway may have a beneficial influence comprising administering a pharmaceutical agent as claimed in claim 48 in an amount sufficient to treat the disease.

52. The method of treating a disease on which modulation of the activity or amount of a protein of the Wnt signal transduction pathway may have a beneficial influence comprising administering a pharmaceutical agent as claimed in claim 49 in an amount sufficient to treat the disease.

53. The method of treating as claimed in claim 50, characterized in that the disease is one selected from the following groups:
the group of cerebral malformations, in particular of cerebral developmental anomalies, cerebral palsies in infants, craniocervical junction abnormalities and dysraphic syndromes,
the group of degenerative and atrophic processes of the brain and the spinal cord, in particular of senile and presenile atrophies of the brain, in particular Alzheimer's disease, Binswanger's disease and Pick's disease,
the group of basal ganglia disorders, in particular Huntington's disease and HDL2, chorea, athetosis and dystonia, spongiform encephalopathies,
the group of degenerations of the corticospinal tract and of the anterior horn of the spinal cord, in particular amyotrophic lateral sclerosis, spinal muscular atrophy and progressive bulbar paralysis,
the group of degenerative ataxias, in particular Friedreich's disease, Refsum's disease and spinocerebellar ataxias type 1-25,
the group of metabolic and toxic processes of the brain and of the spinal cord, Wilson's disease, multiple sclerosis, demyelinating diseases of the central and peripheral nerve system, brain and spinal cord tumors, traumatic damage to the nerve system, circulation disorders of the brain and the spinal cord, in particular hereditary metabolic disorders of the amino acid, lipid, carbohydrate and metal ion metabolisms, in particular Wilson's disease, multiple sclerosis, cerebral infarctions and other forms of stroke, muscular disorders based on damage to the nerve system and post-traumatic muscular atrophies.

54. The method of treating as claimed in claim 51, characterized in that the disease is one selected from the following groups:
the group of cerebral malformations, in particular of cerebral developmental anomalies, cerebral palsies in infants, craniocervical junction abnormalities and dysraphic syndromes,
the group of degenerative and atrophic processes of the brain and the spinal cord, in particular of senile and presenile atrophies of the brain, in particular Alzheimer's disease, Binswanger's disease and Pick's disease,
the group of basal ganglia disorders, in particular Huntington's disease and HDL2, chorea, athetosis and dystonia, spongiform encephalopathies,
the group of degenerations of the corticospinal tract and of the anterior horn of the spinal cord, in particular amyotrophic lateral sclerosis, spinal muscular atrophy and progressive bulbar paralysis,
the group of degenerative ataxias, in particular Friedreich's disease, Refsum's disease and spinocerebellar ataxias type 1-25,
the group of metabolic and toxic processes of the brain and of the spinal cord, Wilson's disease, multiple sclerosis, demyelinating diseases of the central and peripheral nerve system, brain and spinal cord tumors, traumatic damage to the nerve system, circulation disorders of the brain and the spinal cord, in particular hereditary metabolic disorders of the amino acid, lipid, carbohydrate and metal ion metabolisms, in particular Wilson's disease, multiple sclerosis, cerebral infarctions and other forms of stroke, muscular disorders based on damage to the nerve system and post-traumatic muscular atrophies.

55. The method of treating as claimed in claim 52, characterized in that the disease is one selected from the following groups:
the group of cerebral malformations, in particular of cerebral developmental anomalies, cerebral palsies in infants, craniocervical junction abnormalities and dysraphic syndromes,
the group of degenerative and atrophic processes of the brain and the spinal cord, in particular of senile and presenile atrophies of the brain, in particular Alzheimer's disease, Binswanger's disease and Pick's disease,
the group of basal ganglia disorders, in particular Huntington's disease and HDL2, chorea, athetosis and dystonia, spongiform encephalopathies,
the group of degenerations of the corticospinal tract and of the anterior horn of the spinal cord, in particular amyotrophic lateral sclerosis, spinal muscular atrophy and progressive bulbar paralysis,
the group of degenerative ataxias, in particular Friedreich's disease, Refsum's disease and spinocerebellar ataxias type 1-25,
the group of metabolic and toxic processes of the brain and of the spinal cord, Wilson's disease, multiple sclerosis, demyelinating diseases of the central and peripheral nerve system, brain and spinal cord tumors, traumatic damage to the nerve system, circulation disorders of the brain and the spinal cord, in particular hereditary metabolic disorders of the amino acid, lipid, carbohydrate and metal ion metabolisms, in particular Wilson's disease, multiple sclerosis, cerebral infarctions and other forms of stroke, muscular disorders based on damage to the nerve system and post-traumatic muscular atrophies.

56. The method of treating a disease which lead directly or indirectly to the death of brain cells, comprising administering genistein in an amount sufficient to treat the disease.

57. A screening process for detecting brain cell-like cells and brain cells, comprising
(i) determining the concentration of β-catenin, and
(ii) comparing the concentration from (i) with the β-catenin concentration of a suitable comparative cell.

58. The screening process as claimed in claim 57, characterized in that the β-catenin concentration is determined by means of an antibody.

59. The method of use of β-catenin as diagnostic marker for identifying brain cell-like cells and brain cells.

60. An in vitro differentiation of recombinant, neuronal stem cells into brain cell-like cells, effected by a nucleic acid construct for expressing a protein capable of inhibiting a reaction of the Wnt signal transduction pathway.

61. The differentiation as claimed in claim 60, characterized in that the protein is expressed under the control of a constitutive or of a regulatable promoter.

62. The differentiation as claimed in claim 60, characterized in that the cell has been transfected stably or transiently with the nucleic acid construct.

63. A differentiation of a recombinant, neuronal stem cell into brain cell-like cells, effected by at least one protein of the Wnt signal transduction pathway not being expressed, being expressed inactively or being expressed at a reduced level in comparison with the corresponding wild type stem cell.

64. The differentiation as claimed in claim 63, characterized in that at least one gene coding for a protein of the Wnt signal transduction pathway or a DNA section involved in expression of said gene has been completely or partially deleted or has a mutation.

65. A kit for in vitro differentiation of neuronal stem cells and of cells derived from neuronal stem cells, comprising a recombinant, neuronal stem cell which comprises a nucleic acid construct for expressing a protein capable of inhibiting a reaction of the Wnt signal transduction pathway.

66. A kit for in vitro differentiation of neuronal stem cells and of cells derived from neuronal stem cells, comprising a recombinant, neuronal stem cell in which at least one protein of the Wnt signal transduction pathway is not expressed, is expressed inactively or is expressed at a reduced level in comparison with the corresponding wild type stem cell.
